(19) United States
(12) Patent Application Publication
(10) Pub. No.: US 2026/0085051 A1
(43) Pub. Date: Mar. 26, 2026

(54) PREPARATION OF 2-CHLORO-4-FLUORO-5-NITROBENZOIC ACID

(71) Applicant: ADAMA AGAN LTD., Ashdod (IL)

(72) Inventors: Michael GRABARNICK, Metar (IL); Revanappa Vasantrao GALGE, Udgir, Latur, Maharashtra (IN)

(73) Assignee: ADAMA AGAN LTD., Ashdod (IL)

(21) Appl. No.: 19/108,010

(22) PCT Filed: Aug. 31, 2023
(86) PCT No.: PCT/IL2023/050928

§ 371 Date: Feb. 28, 2025

(30) Foreign Application Priority Data
Aug. 31, 2022 (IN) 202211049826

(51) Int. Cl. C07D 239/54; C07C 201/08; C07C 201/16

(52) U.S. Cl. CPC C07D 239/54; C07C 201/08; C07C 201/16

(57) ABSTRACT

The present invention relates to a novel nitration process of 2-chloro-4-fluorobenzoic acid or alkyl 2-chloro-4-fluorobenzoate, using oleum and nitric acid, to obtain 2-chloro-4-fluoro-5-nitrobenzoic acid or alkyl 2-chloro-4-fluoro-5-nitrobenzoates; purifying the crude reaction product; and recovering 2-chloro-4-fluoro-5-nitrobenzoic acid or alkyl 2-chloro-4-fluoro-5-nitrobenzoates in an essentially pure form, which can be used in the synthesis of the herbicide saflufenacil.

Saflufenacil (chemical name: 2-chloro-4-fluoro-5-[3-methyl-2,6-dioxo-4-(trifluoromethyl)-3,6-dihydropyrimidin-1(2H)-yl]-N-[methyl(propan-2-yl)sulfamoyl]benzamide) is an uracil (amide) herbicide. Saflufenacil is a pre-plant and pre-emergence herbicide applied alone or in combination with glyphosate to a wide range of food crops [see PPDB (Pesticide Properties Database created by the University of Hertfordshire https:sitem.herts.ac.uk/aeru/ppdb/en/index.htm)].

Saflufenacil was first described in WO 01/83459. The multistep synthesis of Saflufenacil includes the nitration of 2-chloro-4-fluorobenzoic acid to give 2-chloro-4-fluoro-5-nitrobenzoic acid. The reaction takes place in sulfuric acid, by addition of nitric acid, as reported in Example 1 of WO 01/83459. The reaction illustrated in WO 01/83459 shows 2-chloro-4-fluoro-5-nitrobenzoic acid as the sole reaction product:

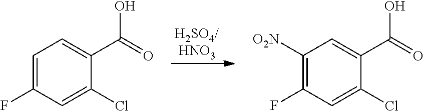 [SMILES: O=C(O)c1ccc(F)cc1Cl ; O=C(O)c1cc([N+](=O)[O-])c(F)cc1Cl]

In fact, the reaction leads to a mixture of isomers, i.e., mononitration occurs at positions 5 or 3 of the aromatic ring:

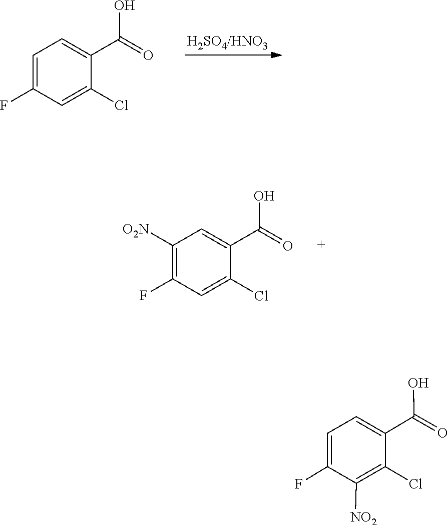 [SMILES: O=C(O)c1ccc(F)cc1Cl ; O=C(O)c1cc([N+](=O)[O-])c(F)cc1Cl ; O=C(O)c1ccc(F)c([N+](=O)[O-])c1Cl]

Hereinafter, 2-chloro-4-fluoro-5-nitrobenzoic acid and 2-chloro-4-fluoro-3-nitrobenzoic acid are referred to as the desired and undesired isomer, respectively. The amount of the undesired isomer is not insignificant, e.g., the crude reaction product usually consists of ˜85-90:10-15 isomeric mixture.

Unfortunately, the undesired isomer is not easily separable from the crude product. Experimental results reported below indicate that purification of a ˜85-90:10-15 isomeric mixture by crystallization to recover the desired isomer can only be achieved with an unacceptably large proportion of solvent and product. Multistep crystallizations were also met with difficulties.

We have found that the conditions of the mononitration reaction of 2-chloro-4-fluorobenzoic acid can be shifted towards the desired isomer in high yields. That is, by using oleum in the nitration reaction, whether on the substituted benzoic acid or on the substituted ester thereof, the desired isomer is preferably produced in 94:6 ratio.

The benefit gained from using oleum in the nitration reaction is due to induced selectivity towards the desired isomer.

The invention is therefore primarily directed to a process comprising the steps of:
reacting 2-chloro-4-fluorobenzoic acid with a nitrating agent prepared from oleum and nitric acid or its salt;
separation of the crude reaction product; and
recovering 2-chloro-4-fluoro-5-nitrobenzoic acid in an essentially pure form;
or to a process of synthesis of alkyl 2-chloro-4-fluoro-5-nitrobenzoates comprising the steps of:
reacting alkyl 2-chloro-4-fluorobenzoate with a nitrating agent prepared from oleum and nitric acid or its salt;
separation of the crude reaction product; and
recovering alkyl 2-chloro-4-fluoro-5-nitrobenzoate in an essentially pure form;
wherein the alkyl contains 1 to 12 carbon atoms.

The reaction is carried under conditions advancing mononitration as a main reaction towards the desired isomer in high yields. A set of conditions enabling efficient management of the mononitration reaction includes the nitrating agent which is prepared from oleum and nitric acid or its salts, reaction temperature, and reaction time. A ratio (based on HPLC analysis, area %) of the undesired isomer and the desired isomer lower than 1:9 is usually satisfactory. A larger proportion of the undesired isomer indicates that loss of yield will take place as a result both of low selectivity and a purification process. Thus, the abovementioned ratio allows maximum yield of the desired isomer. HPLC conditions are described in the experimental section below.

The nitration of 2-chloro-4-fluorobenzoic acid or its alkyl ester is preferably carried out with the oleum/nitric acid combination. The reaction is carried out by feeding more than 1.25 equivalents of nitric acid to the solution of 2-chloro-4-fluorobenzoic acid or its alkyl ester in not less than 2 volumes of oleum at a low temperature.

The nitration of 2-chloro-4-fluorobenzoic acid or its alkyl ester is preferably carried out with the oleum/nitric acid combination. A reaction vessel is charged with oleum 23% solution, followed by the addition of the starting material 2-chloro-4-fluorobenzoic acid or its alkyl ester, at a low temperature.

The addition of nitric acid to the mixture of the oleum 23% with the starting material is carried out gradually with cooling, keeping the temperature during the addition around −10 to +15° C. Preferably, between −5 to +5° C. Yet more preferably, between 0 to 5° C. (the addition is accompanied by the release of heat).

Nitric acid can be supplied to the reaction in the form of 70% to 98% commercially available grades. The oleum 23% solution is a H₂S₂O₇ clear to off white liquid which is a combination of sulfur trioxide in sulfuric acid. Also known as fuming sulfuric acid.

The order of addition can be reversed, and the nitric acid can be added first, for example dropwise, over the oleum, and then the starting material 2-chloro-4-fluorobenzoic acid or its alkyl ester, preferably dropwise at low temperature, e.g. around −10 to +15° C. Preferably, between −5 to +5° C. Yet more preferably, between 0 to 5° C.

Upon completion of the addition of the nitric acid, the reaction mixture may be kept under stirring for about 2 hours. The reaction temperature is usually in the range of 0 to 5° C. Usually, the reaction time is not less than 1 hours, e.g., not less than 2 hours, for example, from 1 to 3 hours (e.g., around 1 to 2 hours).

For example, one variant of the process comprises dissolving 2-chloro-4-fluorobenzoic acid or its alkyl ester in oleum 23%, adding nitric acid at molar excess of at least 25% HNO₃, e.g., of 40% to 80% (around 50%), while cooling the reaction mixture, stirring properly, and maintaining the reaction mixture at said temperature for about 2 hours.

Alternatively, after the above period, a further acid, for example sulfuric acid can be added. In this way, the undesired isomer becomes dinitrated and the separation process becomes more efficient (see example 3). Preferably, the reaction is diluted with the acid, for example sulfuric acid. Typically, the acid is diluted, for example 30-80% v/v acid in water, for example 35-70% v/v.

At the end of the reaction, the crude product in case of the ester is extracted from the aqueous phase with an organic solvent 5 times. After the combination of the organic phases, said one combined organic phase, is washed again with water for two times, followed by evaporation to receive the crude product.

Next, the crude product is purified by recrystallization from an organic solvent, i.e., a solvent, or a mixture of solvents. Purification by recrystallization is not limited to cooling crystallization or evaporation crystallization from a single solvent and includes also the use of solvent pairs. For example, from ethyl acetate/heptane 5-15/85-95 by volume solvent mixture.

Aliphatic hydrocarbons, e.g., heptane, are usually inefficient when used alone but mixtures of an aliphatic hydrocarbon and a medium polarity solvent such as ethyl acetate or isopropanol can enable the isolation of a product with high purity levels. However, in general, higher recovery rates can be achieved by cooling crystallization from alkylated aromatic hydrocarbons, and this technique is generally preferred.

The proportion of an aromatic hydrocarbon solvent and crude product needed to achieve efficient recrystallization is in the range of 3/1 to 10/1, e.g., 4/1 to 9/1, for example, 5/1 to 8/1 (expressed as unit volume of solvent per unit weight of the dried crude product, e.g., mL/g). A typical crude product consists of ˜85-95:5-15 (HPLC, area %) of the desired isomer, and undesired isomer, respectively. By efficient purification we mean achieving an industrially acceptable yield (>75%, e.g., >80%) and low amount of the undesired isomer impurity (<0.5%, e.g., <0.3%)]. The ratio between the solvent and the crude product is expected to vary depending on the type of solvent, targeted purity level and desired recovery rate, and will be adjusted accordingly. For example, toluene (5V to 7V) was shown to be an efficient solvent for recrystallization.

Drying of the recrystallized material can be carried out in vacuo at 45-50° C. for a few hours, to reach a moisture level of not more than 4%. Reducing the moisture level down to, say, 1% is not needed. 2-chloro-4-fluoro-5-nitrobenzoic acid appears to exhibit hygroscopicity to some extent, as it tends to absorb water and restore a water level of ˜4%.

2-chloro-4-fluoro-5-nitrobenzoic acid or its alkyl ester is recovered in an essentially pure form, i.e., free of the undesired isomer, with purity level (by HPLC, area %) of not less than 97.0%, e.g., >98.0%, >99.0%, >99.5%.

The following embodiments and aspects of the present invention will be described:

In one aspect, the present invention provides a process comprising the steps of:
reacting 2-chloro-4-fluorobenzoic acid with a nitrating agent prepared from oleum and nitric acid or its salt;
separation of the crude reaction product; and
recovering 2-chloro-4-fluoro-5-nitrobenzoic acid in an essentially pure form.

In certain embodiments, the process wherein the ratio between oleum volume and mass of 2-chloro-4-fluorobenzoic acid is more than 2.

In certain embodiments, the process wherein the molar excess of nitric acid or its salt at least about 25% relative to 2-chloro-4-fluorobenzoic acid.

In certain embodiments, the process comprising dissolving 2-chloro-4-fluorobenzoic acid in oleum, adding nitric acid or its salt while keeping the reaction mixture cool, and maintaining the reaction mixture at low temperature up to full conversion.

In certain embodiments, the process wherein the crude product is isolated by precipitation upon adding the reaction mixture to cold water and/or ice, following which the crude solid product is separated from the aqueous medium.

In certain embodiments, the process wherein the isolated crude product contains from about 85 to about 95% of 2-chloro-4-fluoro-5-nitrobenzoic acid (HPLC, area %).

In certain embodiments, the process wherein the crude product is purified by recrystallization from one or more solvents.

In certain embodiments, the process wherein the solvent(s) is (are) selected from the group consisting of hydrocarbon solvents, halogenated solvents, ester solvents and ketone solvents or the mixture thereof.

In certain embodiments, the process wherein the ratio between the solvent and the crude product is in the range of 2/1 to 9/1.

In yet another aspect, the present invention provides a process of synthesis of alkyl 2-chloro-4-fluoro-5-nitrobenzoates comprising the steps of:
reacting alkyl 2-chloro-4-fluorobenzoate with a nitrating agent prepared from oleum and nitric acid or its salt;
separation of the crude reaction product; and
recovering alkyl 2-chloro-4-fluoro-5-nitrobenzoate in an essentially pure form.

In certain embodiments, the process wherein the ratio between the oleum volume and the mass of alkyl 2-chloro-4-fluorobenzoate is more than 2.

In certain embodiments, the process wherein the molar excess of nitric acid or its salt at least about 25% relative to the alkyl 2-chloro-4-fluorobenzoate.

In certain embodiments, the process comprising dissolving the alkyl 2-chloro-4-fluorobenzoate in oleum, adding nitric acid or its salt while keeping the reaction mixture cool, and maintaining the reaction mixture at low temperature up to full conversion.

In certain embodiments, the process wherein the crude product is isolated by extraction from the reaction mixture with a chlorinated solvent inert to the oleum or by precipitation upon adding the reaction mixture to cold water and/or ice, following which the crude solid product is separated from the aqueous medium.

In certain embodiments, the process wherein the isolated crude product contains from about 85 to about 95% of alkyl 2-chloro-4-fluoro-5-nitrobenzoate (HPLC, area %).

In certain embodiments, the process wherein the crude product is purified by recrystallization from one or more solvents.

In certain embodiments, the process wherein the solvent(s) is (are) selected from the group consisting of hydrocarbon solvents, halogenated solvents, ester solvents and ketone solvents or the mixture thereof.

In certain embodiments, the process wherein the ratio between the solvent and the crude product is in the range of 2/1 to 9/1.

In certain embodiments, the process wherein the concentration of the oleum is from about 20 to about 70% and is liquid at the reaction conditions.

In certain embodiments, the process further comprising converting 2-chloro-4-fluoro-5-nitrobenzoic acid or its alkyl ester to an herbicidally active compound.

In certain embodiments, the process comprising the steps of: reducing or hydrogenating 2-chloro-4-fluoro-5-nitrobenzoic acid or its alkyl ester to give 5-amino-2-chloro-4-fluorobenzoic acid or its alkyl ester;
transforming these compounds into an ester compound of Formula A1:

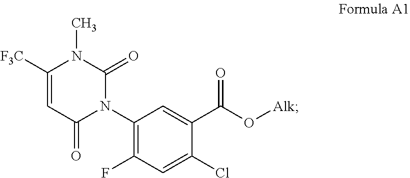

wherein Alk is a C₁₋₁₂ alkyl;
cleaving said ester of Formula A1 to the corresponding benzoic acid of Formula A2:

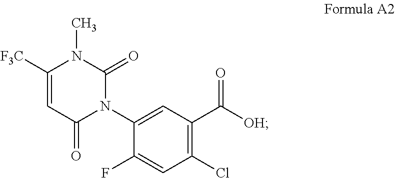 [SMILES: Cn1c(C(F)(F)F)cc(=O)n(-c2cc(C(=O)O)c(Cl)cc2F)c1=O]

and
reacting said benzoic acid A2 with NH₂—SO₂—N[(CH₃)(CH(CH₃)₂)] to afford saflufenacil.

In certain embodiments, the process comprising the steps of:
a) condensing the 2-chloro-4-fluoro-5-nitrobenzoic acid with N-methyl-N-isopropylsulfamoyl amide to give nitro benzoylsulfamide, followed by reduction or hydrogenation of the nitro group to form the amine compound of Formula B1;

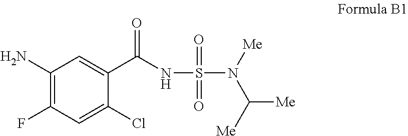 [SMILES: CC(C)N(C)S(=O)(=O)NC(=O)c1cc(N)c(F)cc1Cl]

b) preparing a compound of Formula B2

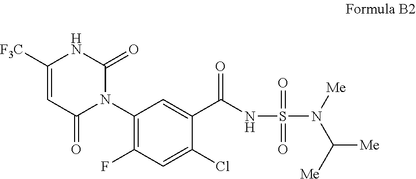 [SMILES: CC(C)N(C)S(=O)(=O)NC(=O)c1cc(-n2c(=O)cc(C(F)(F)F)[nH]c2=O)c(F)cc1Cl]

from the compound of Formula B1 by:
b1) coupling the compound of Formula B1 with 2-dimethylamino-4-(trifluoromethyl)-6H, 1,3-oxazine-6-one; or
b2) converting the compound of Formula B1 to the corresponding isocyanate, and reacting the isocyanate with enamine; or
b3) reacting the compound of Formula B1 with ethyl chloroformate, followed by coupling with an enamine; and
c) methylation of the compound of Formula B2 to afford saflufenacil.

As pointed out above, 2-chloro-4-fluoro-5-nitrobenzoic acid is useful as an intermediate in the synthesis of, inter alia, herbicidally active compounds such as saflufenacil depicted below:

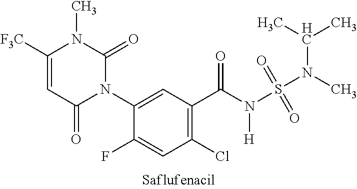 [SMILES: CC(C)N(C)S(=O)(=O)NC(=O)c1cc(-n2c(=O)cc(C(F)(F)F)n(C)c2=O)c(F)cc1Cl]

A process comprising converting the so-formed 2-chloro-4-fluoro-5-nitrobenzoic acid to the herbicidally active compound forms another aspect of the invention.

A few synthetic pathways can be employed to arrive at saflufenacil. For example, 2-chloro-4-fluoro-5-nitrobenzoic acid is reduced or hydrogenated to the corresponding amino compound 2-chloro-4-fluoro-5-aminobenzoic acid, e.g., with the aid of a metal reductant, such as iron or zinc, in an organic solvent, in the presence of an acid. For example, the reaction can take place in acetic acid as a solvent, using iron powder. 2-chloro-4-fluoro-5-aminobenzoic acid is then converted to the intermediate of Formula A1 depicted below, as shown in Examples 3 and 4 of WO 01/83459, through coupling with 2-dimethylamino-4-(trifluoromethyl)-6H, 1,3-oxazine-6-one in acetic acid, followed by alkylation in the presence of a base, e.g., to achieve methylation at the free nitrogen uracil ring and at the acid group (e.g., with methyl iodide and potassium carbonate, in a polar aprotic solvent such as dimethyl formamide)]:

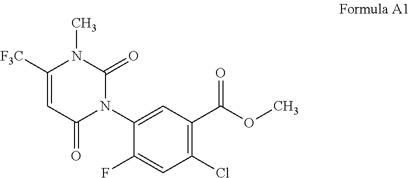 [SMILES: COC(=O)c1cc(-n2c(=O)cc(C(F)(F)F)n(C)c2=O)c(F)cc1Cl]

The ester intermediate of Formula A1 is cleaved to give the corresponding benzoic acid, for example, using BBr₃ as a deprotecting agent. The deprotection reaction takes place in, e.g., methylene chloride. The corresponding benzoic acid thus formed (chemically named 2-chloro-5-(3,6-dihydro-3-methyl-2,6-dioxo-4-(trifluoromethyl)-1(2H)-pyrimidinyl)-4-fluorobenzoic acid, CAS No. 120890-57-5) is collected in the form of a white solid (identified herein as compound of Formula A2):

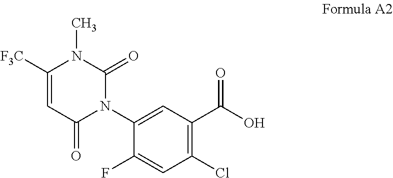 [SMILES: Cn1c(C(F)(F)F)cc(=O)n(-c2cc(C(=O)O)c(Cl)cc2F)c1=O]

The acid reacts with NH₂—SO₂—N[(CH₃)(CH(CH₃)₂)], e.g., to afford saflufenacil. The acid can be activated, by conversion to acid chloride. More convenient, however, is to react the acid with N,N-carbonyldiimidazole (CDI) in tetrahydrofuran at reflux temperature, in the presence of diazabicycloundecane (DBU).

Another important intermediate compound for the synthesis of Saflufenacil is the amino benzoylsulfamide of Formula B1:

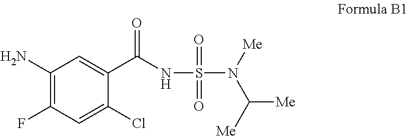 [SMILES: CC(C)N(C)S(=O)(=O)NC(=O)c1cc(N)c(F)cc1Cl]

The preparation of the intermediate of Formula B1 includes condensation of 2-chloro-4-fluoro-5-nitrobenzoic acid with N-methyl-N-isopropylsulfamoyl amide (CAS No. 372136-76-0). The reaction can be carried out in the presence of N,N′-carbonyldiimidazole (CDI) and diazabicycloundecane (DBU) in tetrahydrofuran (THF) as a solvent, as shown in Example 54 of WO 01/83459 to give the corresponding nitro benzoylsulfamide. Reduction of the nitro group as in the previously described route of synthesis, in e.g., acetic acid and THF as cosolvents using iron powder, gives the amino benzoylsulfamide of Formula B1. Also possible is the hydrogenation of the nitro group with Raney nickel in methanol (see hydrogenation procedure in Example 31 of U.S. Pat. No. 7,820,846). A preparation of the intermediate B1 from the acid is illustrated by the reaction scheme depicted below:

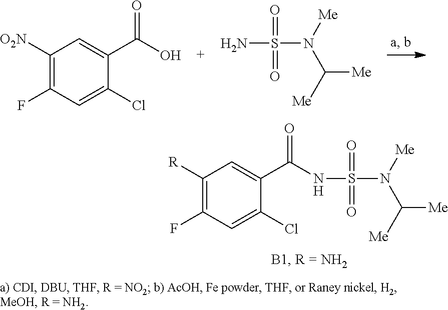

Saflufenacil is accessible from the intermediate of Formula B1 through various routes.

For example, based on WO 01/83459, the coupling of the intermediate of Formula B1 with 2-dimethylamino-4-(trifluoromethyl)-6H, 1,3-oxazine-6-one in acetic acid gave des-methyl saflufenacil of Formula B2:

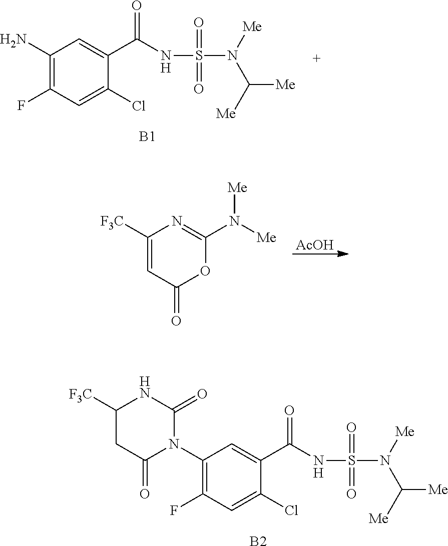

Other approaches involve a cyclization step to form the uracil ring system.

One synthetic route is based on the conversion of the amine group of the intermediate of Formula B1 to isocyanate and coupling it with an ethyl 3-amino-4, 4,4-trifluoro-2-butenoate. The preparation of the phenyl isocyanate is described in U.S. Pat. No. 7,820,846. The amino benzoylsulfamide of Formula B1 was treated with a phosgenating agent to afford the corresponding phenyl isocyanate. Preparation of saflufenacil precursor of Formula B2 (namely, des-methyl saflufenacil) from the isocyanate by coupling it with enamine is described in U.S. Pat. No. 7,737,275. The synthetic route is shown below:

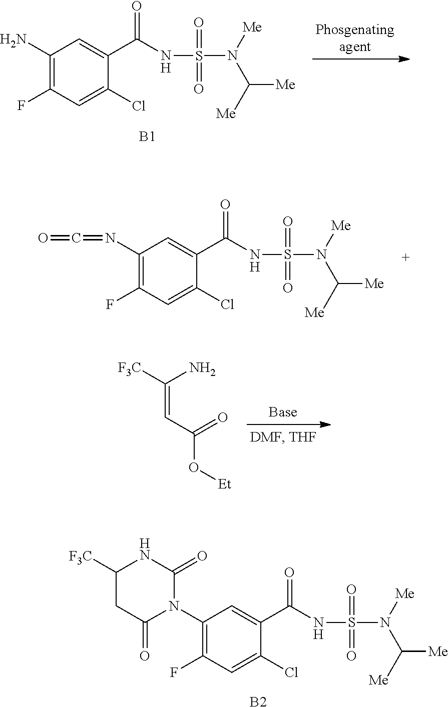

The coupling of the phenyl isocyanate with enamine is performed under an inert atmosphere (water is removed from the reaction vessel by azeotrope drying before the addition of the isocyanatobenzoyl). Different types of bases can be used, e.g., sodium hydride, potassium methoxide, and potassium tert-butoxide as well as potassium carbonate, e.g., MeOK in Example 2 of U.S. Pat. No. 7,737,275.

Another synthetic pathway involving a cyclization step to form the uracil ring system is found in U.S. Pat. No. 8,252,925. The amino benzoylsulfamide intermediate of Formula B1 is treated with a solution of ethyl chloroformate (e.g., in methylene chloride in the presence of pyridine) to give the corresponding ethylate (see Example 1.2 of U.S. Pat. No. 8,252,925). Ring closure is accomplished by reacting the ethylate with ethyl 3-amino-4,4,4-trifluoro-2-butenoate (e.g., in DMF) to form the uracil ring (see Example 3.1.a of U.S. Pat. No. 8,252,925). Resulting des-methyl saflufenacil of Formula B2 is alkylated with dimethyl sulfate (e.g. in toluene/THF) in the presence of an aqueous base, with phase transfer catalyst to afford the targeted active ingredient (see Example 3.3 of U.S. Pat. No. 8,252,925). An exemplary synthetic pathway is depicted below:

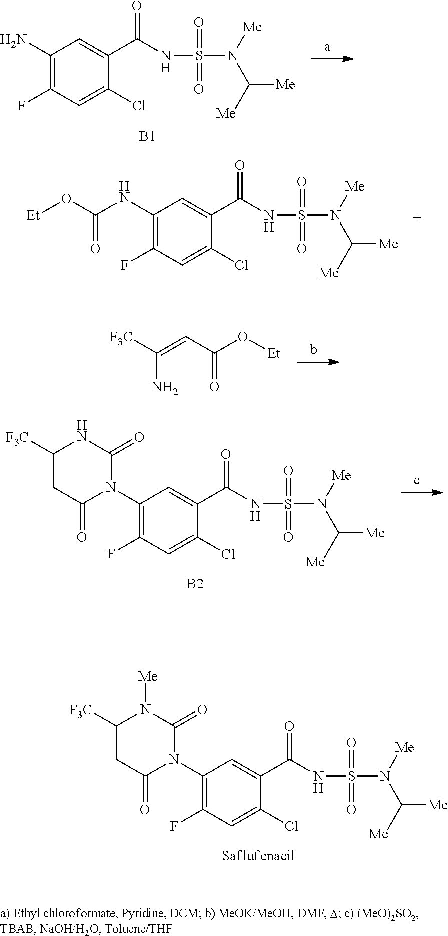

Accordingly, 2-chloro-4-fluoro-5-nitrobenzoic acid prepared according to the invention can be used to produce saflufenacil by processes comprising the following steps:
a) condensing the 2-chloro-4-fluoro-5-nitrobenzoic acid with N-methyl-N-isopropylsulfamoyl amide to give nitro benzoylsulfamide, followed by reduction or hydrogenation of the nitro group to form the amine compound of Formula B1;

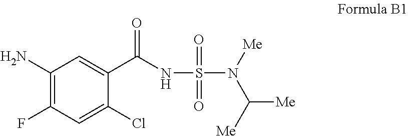 [SMILES: CC(C)N(C)S(=O)(=O)NC(=O)c1cc(N)c(F)cc1Cl]

b) preparing a compound of Formula B2

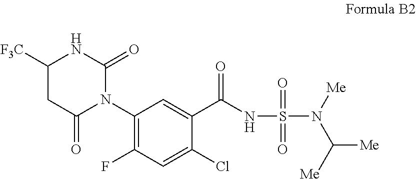 [SMILES: CC(C)N(C)S(=O)(=O)NC(=O)c1cc(N2C(=O)CC(C(F)(F)F)NC2=O)c(F)cc1Cl]

from the compound of Formula B1 by:
b1) coupling the compound of Formula B1 with 2-dimethylamino-4-(trifluoromethyl)-6H, 1,3-oxazine-6-one; or
b2) converting the compound of Formula B1, e.g., with the aid of a phosgenating agent, to isocyanate, and reacting the isocyanate with enamine; or
b3) reacting the compound of Formula B1 with ethyl chloroformate, followed by coupling with an enamine; and
c) methylation of the compound of Formula B2 to afford saflufenacil.

### EXAMPLES

HPLC method conditions: Column: XBridge C18 (4.6×150)mm, 3.5 μm; Mobile phase-A: 10 ammonium bicarbonate in water; Mobile Phase-B: acetonitrile; Gradient (T/% B): 0/10, 10/90, 15/90, 16/10, 20/10; Flow rate: 1 mL/min; Diluent: acetonitrile/water (1:1 v/v); injection volume: 5 μL; run time: 20 minutes). [RRT of undesired isomer 1.22, RRT of dinitro 1.45].

### Example 1

### Preparation and Purification of 2-chloro-4-fluoro-5-nitrobenzoic Acid

8.9 g of 2-chloro-4-fluorobenzoic acid (0.05 mol) were dissolved in 27 mL of oleum 23% and the mixture was cooled to 0° C. To this mixture 4 g of nitric acid 98% were fed while keeping the temperature between 0 to 5° C. with good stirring. The reaction mixture was kept at these conditions for 2 hours and quenched in 300 g of crashed ice. The aqueous mixture was stirred without cooling up to full melting of the ice and the crude product was filtered. The purity of 2-chloro-4-fluoro-5-nitrobenzoic acid was 87 area % with 12.5 area % of the undesirable isomer. Recrystallization of the crude compound from toluene gave the product with a purity of 98 area %.

### Example 2

### Preparation and Purification of Ethyl 2-chloro-4-fluoro-5-nitrobenzoate

10.3 g of ethyl 2-chloro-4-fluorobenzoate (0.05 mol) were dissolved in 30 mL of oleum 23% and the mixture was cooled to 0° C. To this mixture 4 g of nitric acid 98% were fed while keeping the temperature between 0 to 5° C. with good stirring. The reaction mixture was kept at these conditions for 2 hours and extracted with 1,2-dichloroethane 5*70 mL. The organic phases were combined, washed with water 2*50 mL and evaporated giving ethyl 2-chloro-4-fluoro-5-nitrobenzoate with a purity of 89.7 area % and 9.1 area % of the undesirable isomer. Recrystallization of the crude compound from ethyl acetate/heptane gave the product with a purity 98.9 area %.

### Example 3

### Preparation of 2-chloro-4-fluoro-5-nitrobenzoic Acid Followed by Dinitration of Undesired Isomer and Further Purification

Into a 1 L four neck glass RBF equipped with overhead stirrer, temperature probe & condenser 300 ml of 25% Oleum is charged and cooled to 0-5° C. 98% Nitric acid (45.1 g, 0.715 mol, 1.25 eq) is added dropwise at 0-5° C. and the mixture is stirred for 10-15 min at 5-10° C. To this mixture a solution of 2-Chloro-4-Fluorobenzoic acid (100 g, 0.5728 mol, 1.0 eq) in 25% Oleum (200 ml) is added dropwise at 10-15° C., over 3-4 hours and stirred at 10-15° C. for 1 more hour.

At this point the mononitration is complete. To the reaction mixture sulfuric acid (50%, 200 ml) is added dropwise. The addition is exothermic and the temperature raised up to 55-60° C. The mixture was heated to 80-85° C. and maintained at this temperature for 3-5 hours.

Progress of the reaction is monitored by HPLC analysis according to the area %.

After completion of reaction, the reaction mass is cooled to 30° C. and quenched by adding slowly to ice+water (1500 ml) while maintaining the temperature below 30° C.

The reaction mass is stirred for 1-2 hours at 25-30° C., filtered and the solid washed with water (100 ml) furnishing wet cake (228 g) and filtrate MLR (2765 g). Wet cake is 2-Chloro-4-fluoro-5-nitrobenzoic acid with purity 91% and content of 2-Chloro-4-fluoro-3-nitrobenzoic acid 3.3% and of 2-Chloro-4-fluoro-3,5-dinitrobenzoic acid 4.3%.

To the 500 mL four neck RFB equipped with overhead stirrer, temperature probe & condenser xylene (500 ml) and wet cake (228 g) is charged. The content is heated gradually to 85-90° C. up to full dissolution of the solid and aqueous phase (91 g) is separated from organic layer (548 g).

The organic layer is cooled to 25-30° C. and stirred for 1-2 hours at 25-30° C. The solid is filtered and the wet cake (160 g) dried at 40-45° C. for 6-8 hours. Yield of 2-Chloro-4-fluoro-5-nitrobenzoic acid 112 g, 89% with purity 99.1%.

1) A process comprising the steps of:
reacting 2-chloro-4-fluorobenzoic acid with a nitrating agent prepared from oleum and nitric acid or its salt;
separation of the crude reaction product; and
recovering 2-chloro-4-fluoro-5-nitrobenzoic acid in an essentially pure form.

2) A process according to claim 1, wherein the ratio between oleum volume and 2-chloro-4-fluorobenzoic acid is more than 2.

3) A process according to claim 1 or 2, wherein the molar excess of nitric acid or its salt at least about 25% relative to 2-chloro-4-fluorobenzoic acid.

4) A process according to claim 1, comprising dissolving 2-chloro-4-fluorobenzoic acid in oleum, adding nitric acid or its salt while keeping the reaction mixture cool, and maintaining the reaction mixture at low temperature up to full conversion.

5) A process according to any one of claims 1 to 4, wherein the crude product is isolated by precipitation upon adding the reaction mixture to cold water and/or ice, following which the crude solid product is separated from the aqueous medium.

6) A process according to claim 5, wherein the isolated crude product contains from about 85 to about 95% of 2-chloro-4-fluoro-5-nitrobenzoic acid (HPLC, area %).

7) A process according to any one of claims 1 to 6, wherein the crude product is purified by recrystallization from one or more solvents.

8) A process according to claim 7, wherein the solvent(s) is (are) selected from the group consisting of hydrocarbon solvents, halogenated solvents, ester solvents and ketone solvents or the mixture thereof.

9) A process according to claim 8, wherein the ratio between the solvent and the crude product is in the range of 2/1 to 9/1.

10) A process of synthesis of alkyl 2-chloro-4-fluoro-5-nitrobenzoates comprising the steps of:
reacting alkyl 2-chloro-4-fluorobenzoate with a nitrating agent prepared from oleum and nitric acid or its salt;
separation of the crude reaction product; and
recovering alkyl 2-chloro-4-fluoro-5-nitrobenzoate in an essentially pure form.

11) A process according to claim 10, wherein the ratio between oleum volume and mass of 2-chloro-4-fluorobenzoic acid is more than 2.

12) A process according to claims 10 or 11, wherein the molar excess of nitric acid or its salt at least about 25% relative to the alkyl 2-chloro-4-fluorobenzoate.

13) A process according to claim 12, comprising dissolving the alkyl 2-chloro-4-fluorobenzoate in oleum, adding nitric acid or its salt while keeping the reaction mixture cool, and maintaining the reaction mixture at low temperature up to full conversion.

14) A process according to any one of claims 10 to 13, wherein the crude product is isolated by extraction from the reaction mixture with a chlorinated solvent inert to the oleum or by precipitation upon adding the reaction mixture to cold water and/or ice, following which the crude solid product is separated from the aqueous medium.

15) A process according to claim 14, wherein the isolated crude product contains from about 85 to about 95% of alkyl 2-chloro-4-fluoro-5-nitrobenzoate (HPLC, area %).

16) A process according to any one of claims 10 to 15, wherein the crude product is purified by recrystallization from one or more solvents.

17) A process according to claim 16, wherein the solvent(s) is (are) selected from the group consisting of hydrocarbon solvents, halogenated solvents, ester solvents and ketone solvents or the mixture thereof.

18) A process according to claim 17, wherein the ratio between the solvent and the crude product is in the range of 2/1 to 9/1.

19) A process according to claims 1 or 10, wherein the concentration of the oleum is from about 20 to about 70% and is liquid at the reaction conditions.

20) A process according to any one of the preceding claims, further comprising converting 2-chloro-4-fluoro-5-nitrobenzoic acid or its alkyl ester to an herbicidally active compound.

21) A process according to claim 20, comprising the steps of:
reducing or hydrogenating 2-chloro-4-fluoro-5-nitrobenzoic acid or its alkyl ester to give 5-amino-2-chloro-4-fluorobenzoic acid or its alkyl ester;
transforming these compounds into an ester compound of Formula A1: 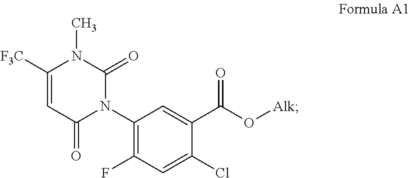
wherein Alk is a C₁₋₁₂ alkyl;
cleaving said ester of Formula A1 to the corresponding benzoic acid of Formula A2: 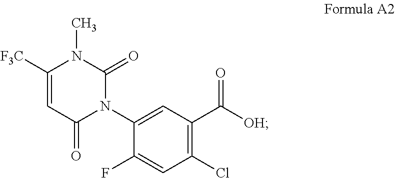 [SMILES: Cn1c(C(F)(F)F)cc(=O)n(-c2cc(C(=O)O)c(Cl)cc2F)c1=O]
and
reacting said benzoic acid A2 with NH₂—SO₂—N[(CH₃)(CH(CH₃)₂)] to afford saflufenacil.

22) A process according to claim 20, comprising the steps of:
a) condensing the 2-chloro-4-fluoro-5-nitrobenzoic acid with N-methyl-N-isopropylsulfamoyl amide to give nitro benzoylsulfamide, followed by reduction or hydrogenation of the nitro group to form the amine compound of Formula B1; 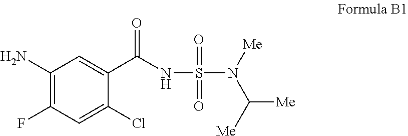 [SMILES: CC(C)N(C)S(=O)(=O)NC(=O)c1cc(N)c(F)cc1Cl]
b) preparing a compound of Formula B2 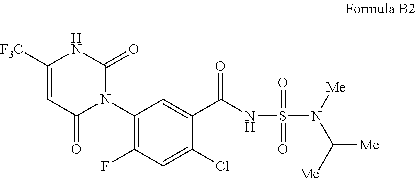 [SMILES: CC(C)N(C)S(=O)(=O)NC(=O)c1cc(-n2c(=O)cc(C(F)(F)F)[nH]c2=O)c(F)cc1Cl]
from the compound of Formula B1 by:
b1) coupling the compound of Formula B1 with 2-dimethylamino-4-(trifluoromethyl)-6H, 1,3-oxazine-6-one; or
b2) converting the compound of Formula B1 to the corresponding isocyanate, and reacting the isocyanate with enamine; or
b3) reacting the compound of Formula B1 with ethyl chloroformate, followed by coupling with an enamine; and
c) methylation of the compound of Formula B2 to afford saflufenacil.
